(19) United States
(12) Patent Application Publication
(10) Pub. No.: US 2010/0065659 A1
(43) Pub. Date: Mar. 18, 2010

(54) Air pop dancing light air freshener

(76) Inventor: Bill Nguyen, Carson, CA (US)

Correspondence Address: BILL NGUYEN, 16536 SAN PEDRO ST., CARSON, CA, 90746, US

(21) Appl. No.: 12/586,062

(22) Filed: Sep. 15, 2009

Related U.S. Application Data
(60) Provisional application No. 61/192,111, filed on Sep. 16, 2008.

(51) Int. Cl. A61L 9/00

(52) U.S. Cl. 239/289

(57) ABSTRACT

An air pop dancing light-air freshener comprising: A housing base, an internal or an external electric air pump. Above the housing base are two crystal clear panels walls that create a very thin gap about 0.015 to 0.035 of an inch apart. Between the panels walls, one would pour the scented liquid. The air pump forces air between the two panels wall and through the scented liquid, create the “dancing” motion of the air pop and scented liquid, This action also disperses the scent throughout the room. The air pop dancing light can be easily refilled with various scented liquid.

### BACKGROUND OF THE INVENTION

This present invention relates to the field of room air fresheners and more particularly to air freshener having, an air pump forces air in to an air tank and forces compressed air extreme fast between the two clear panels walls and through the scented liquid, creating the dancing waved motion of the air and the scented liquid. This action also disperses the scent throughout the room for household or anywhere else where it is need to freshen the air.

### SUMMARY OF THE INVENTION

The present invention is to providing an air freshener and entertainment device. The air pop dancing light is an air freshener that housing an electric air pump in the base, above the base are two crystal clear panels that create a very thin gap. Between the crystal clear panels walls, one would pour the scent liquid.

The air pump forces air between the two crystal clear panels walls and through the scent liquid, create the “dancing” motion of the compressed air and liquid. This action also disperses the scent throughout the room.

Another object of the present invention is to provide the back-light for emitting the Soft light throughout a dark room.

Another object of the present invention is to provide, can be easily refilled with various scented liquid.

### BRIEF DESCRIPTION OF DRAWINGS

FIG. 1 is an Isometric view of an air pop dancing light air freshener 1, LED lights 5, and the scented liquid 8, in power off state.

FIG. 2 is an Isometric view of an air pop dancing light air freshener 1, LED lights 5, and the scented liquid 8, in power on state.

FIG. 3 is an Isometric view of an air pop dancing light air freshener 1, rectangle bar light 5B, and the scented liquid 8, in power on state.

FIG. 4 is a cross-sectional view of an air pop dancing light air freshener 1.

### DETAILED DESCRIPTION OF THE INVENTION

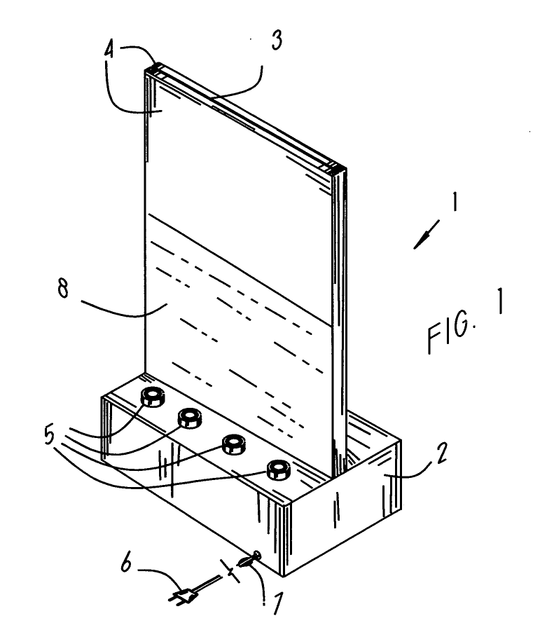

As shown in. FIG. 1 schematically illustrates an air pop dancing light air freshener 1, comprises: a housing base 2, a very thin gap 3, about 0.015 to 0.035 of an inch apart. between the panel's walls 4, one would pour the scented liquid 8 in power off state, the LED lights 5, an electrical plug 6, an electrical switch 7.

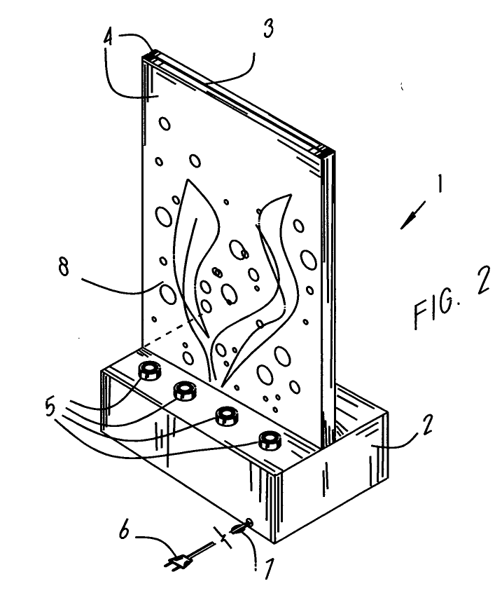

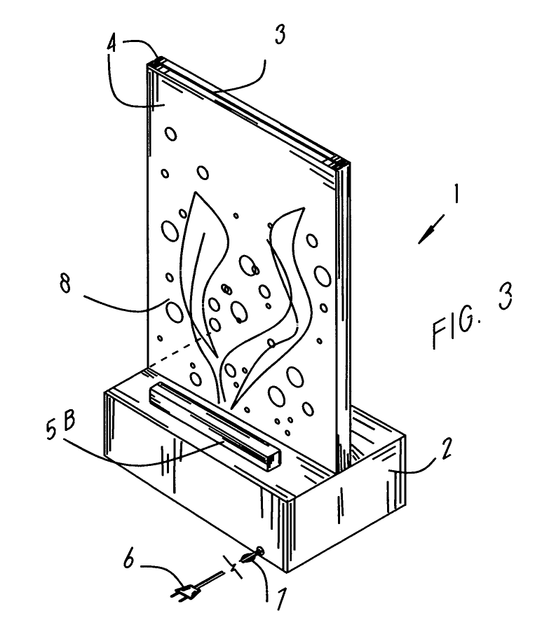

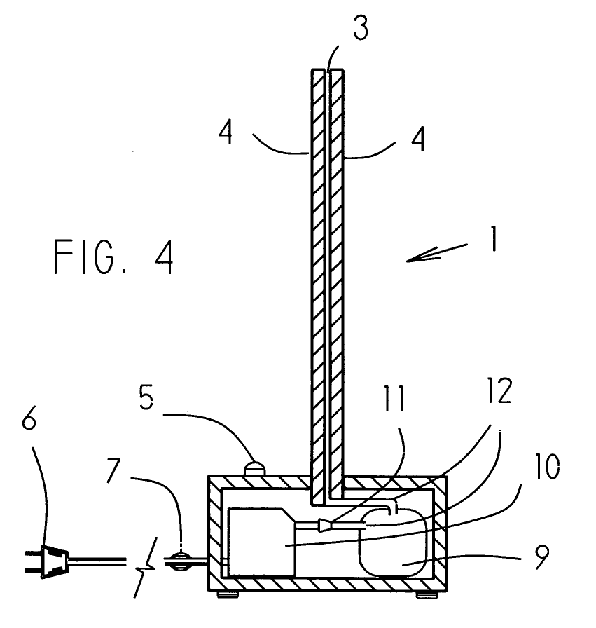

FIG. 2 to FIG. 4 schematically illustrates an air pop dancing light air freshener 1, comprises: a compressing air tank 9, an electric air pump 10, a check valve 11, the air lines 12. The air pump forces air between the two panels walls 4, and through the scented liquid 8, create the “dancing” motion of the air pop and scented liquid, this action also disperses the scent liquid emitting the fragrance from the very thin gap 3, throughout the room.

What is claimed is:

1. An air pop dancing light air freshener comprising:
A housing base box having an internal or an external electric air pump, a check valve, an air tank, above the housing base box having two crystal panels Stand-up that create a very thin parallel gap between walls and the gap of both side is sealed, the bottom side gap is not seal at the center where to let the forced air into the longitude gap and the top side gap is open for scent out-let, multiple LED lights on top of the housing box or a rectangle light bar on top of the housing box.
